(19) United States
(12) Patent Application Publication
(10) Pub. No.: US 2018/0207094 A1
(43) Pub. Date: Jul. 26, 2018

(54) IMPROVED TOPICAL KETOPROFEN FORMULATIONS

(71) Applicant: Elliptical Therapeutics, LLC, San Diego, CA (US)

(72) Inventors: Samir Roy, San Diego, CA (US); Charlotte P. Clark, Keizer, OR (US)

(21) Appl. No.: 15/742,708

(22) PCT Filed: Jun. 30, 2016
(86) PCT No.: PCT/US16/40315

§ 371 Date: Jan. 8, 2018

Related U.S. Application Data
(60) Provisional application No. 62/189,891, filed on Jul. 8, 2015.

(51) Int. Cl. A61K 9/06; A61K 9/00; A61K 31/192; A61K 47/10; A61K 47/12; A61K 47/14; A61K 47/18; A61K 47/26; A61K 47/44; A61P 29/00

(52) U.S. Cl. CPC A61K 9/06; A61K 9/0014; A61K 31/192; A61K 47/10; A61P 29/00; A61K 47/14; A61K 47/18; A61K 47/26; A61K 47/44; A61K 47/12

(57) ABSTRACT

A topical formulation comprising an oil-in-water (o/w) emulsion of ketoprofen in a racemic or (s)-enantiomer form is provided herein. In some embodiments, the topical formulation is a cream. In some embodiments, the topical formulation includes one or more oily substances, higher alcohols, drug solubilizing vehicles, surfactants, permeation enhancers, buffering agents, antioxidants, preservatives, and water.

### CROSS-REFERENCE TO RELATED APPLICATIONS

This application claims priority to U.S. Provisional Patent Application Ser. No. 62/189,891, filed Jul. 8, 2015, which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates to pharmaceutical formulations and specifically to topical formulations comprising ketoprofen, which may be used to treat inflammation and/or pain.

### BACKGROUND

Ketoprofen (C₁₆H₁₄O₃) is a nonsteroidal agent with anti-inflammatory, analgesic, and antipyretic activities. It acts by inhibiting cyclooxygenase-1 and -2 (COX-1 and COX-2) enzymes reversibly, thereby decreasing production of inflammatory-inducing prostaglandin precursors. Ketoprofen has been used clinically to alleviate pain and inflammation associated with inflammatory conditions, such as mild to moderate arthritis.

When ketoprofen is administered orally, for example, as a capsule or tablet, common systemic side effects, including gastric irritation, ulcers, renal impairment, and hepatotoxicity, have been observed. The incidence of such severe common adverse reactions has limited the use of ketoprofen for an extended period of time.

To overcome these clinical adverse events, several topical formulations have been developed for local pain management, for example, as described in U.S. Pat. No. 4,534,980 to Itoh et al. and U.S. Pat. No. 8,822,537 to Buyuktimkin et al, the disclosures of which are herein incorporated by reference in their entireties. Previously developed topical formulations typically have a drug loading of 5-10% w/w. Some of these formulations, such as the formulations disclosed in Buyuktimkin, contain lower alcohols such as ethanol, propylene glycol, glycerin, etc. to achieve the desired skin permeability. These lower alcohols are likely to chemically interact with ketoprofen to form esters, thereby leading to formulation instability and shortened product shelf-life. Other formulations, such as those described by Itoh, include crotamiton as an anti-nucleating agent in an effort to prevent crystallization and extend shelf-life. Despite the inclusion of crotamiton, crystals may still form due to storage temperature fluctuations.

Accordingly, there is a need for new and useful ketoprofen formulations with improved stability. There is also a need for improved ketoprofen formulations with reduced side effects, because rashes and itching have been commonly reported with existing topical formulations. The present disclosure provides such new and useful formulations.

### SUMMARY

The present disclosure is directed to a topical ketoprofen formulation. One aspect of the disclosure is directed to a topical ketoprofen formulation with improved chemical and physical stability, as compared to existing formulations. Another aspect of the disclosure is directed to a topical ketoprofen formulation with reduced skin irritation potential, as compared to existing formulations. Another aspect of the disclosure is directed to a composition formulated to enhance the skin permeability of ketoprofen using penetration enhancers to achieve efficacious drug concentration at the site of action. Another aspect of the disclosure is directed to cream ketoprofen formulations. Another aspect of the disclosure is directed to a method of applying an improved ketoprofen formulation for the treatment of inflammation and/or pain associated with arthritis.

One aspect of the disclosure is directed to a topical composition, which comprises, on a weight basis, about 2% to about 20% of ketoprofen, about 5% to about 20% of one or more oily substances, about 1% to about 20% of one or more higher alcohols, about 1 to about 10% of one or more permeation enhancers, about 0.01% to about 0.5% of one or more preservatives, about 0.1% to about 5% of one or more buffering agents or pH controlling agents, and about 45% to about 70% of purified water. In some embodiments, the topical composition additionally includes about 1% to about 10% by weight of one or more surfactants. In some embodiments, the topical composition additionally or alternatively includes about 0.1% to about 2% by weight of one or more antioxidants.

Another aspect of the disclosure is directed to a topical composition which comprises, on a weight basis: 5 to 15% ketoprofen; 5 to 15% of an oily substance; 5 to 15% of a higher alcohol; 0.02 to 0.1% of an antioxidant; 5 to 15% of a surfactant or emulsifying agent; 5 to 15% of solubilizing vehicle; 1 to 10% of a permeation enhancer; 0.01 to 0.5% of a preservative; 1 to 5% of a buffering agent; and 40 to 60% of purified water. In some embodiments, the topical composition is a cream.

In some embodiments, the ketoprofen is present substantially in (s)-enantiomeric form. In some embodiments, the ketoprofen is present in a racemic mixture. In some embodiments, the antioxidant comprises one or more of: tocopherol and butylated hydroxytoluene. In some embodiments, the drug solubilizing vehicle comprises one more of: isopropyl alcohol, low molecular weight polyethylene glycol, and isobutyl alcohol. In some embodiments, the permeation enhancer comprises one or more of: propylene glycol monolaurate, isopropyl myristate, and oleic acid. In some embodiments, the higher alcohol acts as a stiffening agent and comprises one or more of: cetyl alcohol and stearyl alcohol. In some embodiments, the preservative comprises one or both of methyl paraben and propyl paraben. In some embodiments, the buffering agent comprises one or more of: epolamine, triethanolamine, tromethamine, and diethanolamine. In some embodiments, the surfactant comprises one or more of: polyoxyethylene monostearate, glyceryl monostearate, and glyceryl monooleate.

In some embodiments, the higher alcohol exists in an oleaginous phase comprising one or more hydrocarbons selected from the group consisting of: liquid paraffin, white petrolatum, bees wax, peanut oil, sesame oil, and soybean oil.

In some embodiments, the purified water is in an aqueous phase.

In some embodiments, the pH of the composition ranges from 3.5 to 7.5. In some embodiments, the pH of the composition ranges from 4.5 to 5.5.

In some embodiments, the oily substance comprises one or more of: liquid paraffin, white petrolatum, peanut oil, sesame oil, soybean oil, bees wax, and synthetic oil.

In some embodiments, the topical composition is used to treat an individual with an inflammatory condition. In some embodiments, the topical composition is used to treat an individual with arthritis. In some embodiments, the topical composition is used to treat an individual with pain.

### BRIEF DESCRIPTION OF THE DRAWINGS

FIG. 1 shows a Franz cell diffusion system to assess ketoprofen cream formulation skin permeation.

FIG. 2A shows a histogram depicting a cumulative amount (μg/cm²) over time (hours) of ketoprofen cream formulation (KPC-C and KPC-D) that permeated cadaver skin in the Franz cell diffusion system of FIG. 1.

FIG. 2B shows a histogram depicting a cumulative amount (μg/cm²) over time (hours) of ketoprofen cream formulation (KPC-E and KPC-F) that permeated cadaver skin in the Franz cell diffusion system of FIG. 1.

FIG. 3 shows a line graph depicting a percent label strength over time (months) of ketoprofen cream formulations (KPC-E and KPC-F) stored at 25° C.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the invention to these embodiments, but rather to enable any person skilled in the art to make and use this invention. Other embodiments may be utilized and modifications may be made without departing from the spirit or the scope of the subject matter presented herein. Aspects of the disclosure, as described and illustrated herein, can be arranged, combined, modified, and designed in a variety of different formulations, all of which are explicitly contemplated and form part of this disclosure.

Unless otherwise defined, each technical or scientific term used herein has the same meaning as commonly understood by one of ordinary skill in the art to which this disclosure belongs.

As used in the description and claims, the singular form “a”, “an” and “the” include both singular and plural references unless the context clearly dictates otherwise. For example, the term “an oily substance” may include, and is contemplated to include, a plurality of oily substances. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a percent by weight), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a substance or composition.

As used herein, the term “comprising” or “comprises” is intended to mean that the composition or formulation includes the recited elements, and may additionally include any other elements. “Consisting essentially of” shall mean that the composition or formulation includes the recited elements and excludes other elements of essential significance to the combination for the stated purpose. Thus, a formulation consisting essentially of the elements as defined herein would not exclude other compounds or substances that do not materially affect the basic and novel characteristic(s) of the claimed invention. “Consisting of” shall mean that the composition or formulation includes the recited elements and excludes anything more than trivial or inconsequential elements. Embodiments defined by each of these transitional terms are within the scope of this disclosure.

Disclosed herein are pharmaceutical compositions formulated for topical application. In various embodiments, the compositions are formulated for treating one or more of inflammation and pain. Some embodiments are formulated for acute use while other embodiments are formulated for chronic use, for example, for the treatment of chronic pain or inflammation associated with arthritis or other chronic conditions. In some embodiments, the topical formulation is a cream. In other embodiments, the topical formulation is an ointment, lotion, liniment, or gel. The various formulations described herein include a therapeutically effective amount of ketoprofen.

Ketoprofen is a chiral drug and exists as an equal mixture of S and R enantiomers in a racemic mixture. It is reported that essentially all of the pharmacological activity resides in the S-enantiomer, which is approximately twice as potent as the racemate. In contrast, the R-enantiomer has little or no anti-inflammatory and antipyretic activities. Accordingly, some embodiments provided herein are directed to a ketoprofen formulation containing only the S-enantiomer of ketoprofen. Such a formulation would require half as much ketoprofen as the racemic mixture to elicit equivalent pharmacological effects and would significantly reduce skin irritation.

In some embodiments, the topical formulation is an oil-in-water (o/w) emulsion comprising the active ingredient ketoprofen in a racemic or (s)-enantiomer form. In some such embodiments, the topical formulation is a semi-solid or viscous liquid having the consistency of a cream. In some embodiments, the cream or other topical formulation contains one or more oily substances, higher alcohols, surfactants, permeation enhancers, buffering agents, drug solubilizing vehicles, antioxidants, preservatives, and water. The topical formulation of some embodiments comprises: 2-20% by weight ketoprofen (racemic or (s)-enantiomer); 5-20% by weight of oily substances; 0.1-2% by weight antioxidant; 1-20% by weight of higher alcohol; 1 to 5% by weight permeation enhancers; 1 to 15% by weight surfactants; 0.01 to 0.5% by weight preservatives; 1 to 5% by weight pH controlling agent or buffer; 1 to 30% by weight drug solubilizing vehicle; and 40 to 70% by weight purified water.

In one embodiment, the topical formulation comprises: 5-15%, by weight ketoprofen (racemic or (s)-enantiomer); 10-15%, by weight of oily substances; 0.5-1%, by weight antioxidant; 10-15%, by weight of higher alcohol; 2-4%, by weight permeation enhancers; 7-10%, by weight surfactants; 0.05-0.2%, by weight preservatives; 2-4%, by weight pH controlling agent or buffer; 5 to 15% by weight drug solubilizing vehicle; and 50-65%, by weight purified water.

In one embodiment, the topical formulation comprises by weight: 5% ketoprofen, 5% mineral oil, 5% white petrolatum, 10% cetyl alcohol and stearyl alcohol mixture, 4% polyoxyethylene monostearate, 4% glyceryl monostearate, 8% isopropyl alcohol, 2% isopropyl myristate, 0.1% methyl paraben, 0.02% propyl paraben, 0.05% butylated hydroxytoluene, 1% triethanolaine, and 55.8% water.

In one embodiment, the topical formulation comprises by weight: 9% ketoprofen, 5% mineral oil, 5% white petrolatum, 10% cetyl alcohol and stearyl alcohol mixture, 4% polyoxyethylene monostearate, 4% glyceryl monostearate, 8% isopropyl alcohol, 2% isopropyl myristate, 0.1% methyl paraben, 0.02% propyl paraben, 0.05% butylated hydroxytoluene, 1.5% triethanolaine, and 51.3% water.

In one embodiment, the topical formulation comprises by weight: 12% ketoprofen, 5% mineral oil, 5% white petrolatum, 10% cetyl alcohol and stearyl alcohol mixture, 4% polyoxyethylene monostearate, 4% glyceryl monostearate, 8% isopropyl alcohol, 2% isopropyl myristate, 0.1% methyl paraben, 0.02% propyl paraben, 0.05% butylated hydroxytoluene, 2% triethanolaine, and 57.8% water.

In one embodiment, the topical formulation comprises by weight: 10% ketoprofen, 5% mineral oil, 5% white petrolatum, 10% cetyl alcohol and stearyl alcohol mixture, 4% polyoxyethylene monostearate, 4% glyceryl monostearate, 10% isopropyl alcohol, 2% isopropyl myristate, 2% oleic acid, 0.1% methyl paraben, 0.02% propyl paraben, 0.05% butylated hydroxytoluene, 2.5% triethanolaine, and 45.3% water.

In one embodiment, the topical formulation comprises by weight: 10% ketoprofen, 5% mineral oil, 5% white petrolatum, 10% cetyl alcohol and stearyl alcohol mixture, 4% polyoxyethylene monostearate, 4% glyceryl monostearate, 8% isopropyl alcohol, 2% isopropyl myristate, 0.1% methyl paraben, 0.02% propyl paraben, 0.05% butylated hydroxytoluene, 2.5% epolamine, and 47.3% water.

In some embodiments, the 10% cetyl alcohol and stearyl alcohol mixture includes 0% cetyl alcohol and 100% stearyl alcohol; 5% cetyl alcohol and 95% stearyl alcohol; 10% cetyl alcohol and 90% stearyl alcohol; 15% cetyl alcohol and 85% stearyl alcohol; 20% cetyl alcohol and 80% stearyl alcohol; 25% cetyl alcohol and 75% stearyl alcohol; 30% cetyl alcohol and 70% stearyl alcohol; 35% cetyl alcohol and 65% stearyl alcohol; 40% cetyl alcohol and 60% stearyl alcohol; 45% cetyl alcohol and 55% stearyl alcohol; 50% cetyl alcohol and 50% stearyl alcohol; 55% cetyl alcohol and 45% stearyl alcohol; 60% cetyl alcohol and 40% stearyl alcohol; 65% cetyl alcohol and 35% stearyl alcohol; 70% cetyl alcohol and 30% stearyl alcohol; 75% cetyl alcohol and 25% stearyl alcohol; 80% cetyl alcohol and 20% stearyl alcohol; 85% cetyl alcohol and 15% stearyl alcohol; 90% cetyl alcohol and 10% stearyl alcohol; 95% cetyl alcohol and 55% stearyl alcohol; or 100% cetyl alcohol and 0% stearyl alcohol.

In another embodiment, the cream or other topical composition includes, on a weight basis, about 2 to about 20 percent ketoprofen, about 5 to about 20% of an oily substance, about 1 to about 20% of a higher alcohol, about 0.1 to about 2% of an antioxidant, about 5 to about 15% of a surfactant or emulsifying agent, about 1 to about 10% of a permeation enhancer, about 0.01 to about 0.5% of a preservative, about 0.1 to about 5% of a buffering agent, about 1 to 25% drug solubilizing vehicle, and about 40 to about 70% of purified water. In some such embodiments, only the (s)-enantiomer form of ketoprofen is provided in the formulation. In other embodiments, the formulation includes a racemic mixture of the active ingredient.

In some embodiments, the antioxidant is tocopherol (Vitamin E), butylated hydroxytoluene (BHT), or a combination thereof. In some embodiments, Vitamin E and/or BHT functions as an antioxidant at the location of topical application.

In some embodiments, a drug solubilizing vehicle is ethyl alcohol, isopropyl alcohol, butyl alcohol, propylene glycol, polyethylene glycol, low molecular weight polyethylene glycol, isobutyl alcohol, or a combination of one or more thereof. The lower alcohols assist in solubilizing the drug at high loading in the cream and also have synergistic effects with other permeation enhancers.

In some embodiments, the permeation enhancer is propylene glycol monolaurate, isopropyl myristate, oleic acid, or a combination of one or more thereof. The permeation enhancer of various embodiments functions to facilitate permeation of the active drug into skin. In various embodiments, the permeation enhancer is present in a sufficiently small amount (for example, less than 5% by weight) so as to not significantly affect the stability of the formulation.

In some embodiments, the preservative is methyl paraben, propyl paraben, or a combination thereof. The preservatives of various embodiments improve shelf-life of the formulation and/or prevent microbial growth.

In some embodiments, the buffering agent is epolamine, triethanolamine, diethanolamine, tromethamine, or a combination of one or more of epolamine, triethanolamine, diethanolamine, and tromethamine. In various embodiments, the buffering agent adjusts, buffers, and controls the pH of the formulation, neutralizing fatty acids and solubilizing oils and other non-water soluble ingredients.

In some embodiments, the surfactant is polyoxyethylene monostearate, glyceryl monostearate, glyceryl monooleate, or a combination of one or more of polyoxyethylene monostearate, glyceryl monostearate, and glyceryl monooleate. The surfactant of various embodiments acts as an emulsifier, lowering the surface tension between the oils and water in the formulation.

In some embodiments, the oily substance is liquid paraffin, mineral oil, white petrolatum, bees wax, peanut oil, sesame oil, soybean oil, other plant oil, synthetic oil, or other oily substance used as an oleaginous vehicle for a cream.

In some embodiments, the higher alcohol is stearyl alcohol, cetyl alcohol, or other higher alcohol used as a stiffening agent for the cream composition. The higher alcohols of various embodiments do not react with ketoprofen. The use of higher alcohols rather than lower alcohols reduces or eliminates the formation of ester degradation products, thereby improving the chemical stability of the ketoprofen formulation. Moreover, by stiffening the composition into a cream, there is little to no phase separation between oil and water during storage; accordingly, in various embodiments, the ketoprofen cream formulation is physically stable.

In some embodiments, the hydrocarbons are in an oleaginous phase. In some embodiments, the purified water is in an aqueous phase.

In some embodiments, the pH of the ketoprofen cream formulation ranges from about 3.5 to about 7.5. In some embodiments, the pH of the ketoprofen cream formulation ranges from about 4.5 to about 5.5. In some embodiments, the pH of the ketoprofen cream formulation ranges from about 5 to about 7. In one embodiment, the pH of the ketoprofen cream formulation is 5.0. In one embodiment, the pH of the ketoprofen cream formulation is 5.1. In one embodiment, the pH of the ketoprofen cream formulation is 5.2. In one embodiment, the pH of the ketoprofen cream formulation is 5.3. In one embodiment, the pH of the ketoprofen cream formulation is 5.4. In one embodiment, the pH of the ketoprofen cream formulation is 5.5.

In some embodiments, the provided topical ketoprofen composition is formulated to reduce or alleviate local pain, for example, pain associated with one or more joints, such as the ankles, knees, shoulders, elbows, hips, or joints of the finger, pain associated with carpal tunnel, tennis elbow, or other strain or sprain of a ligament or tendon, or pain associated with a contusion, inflammation, or other tissue injury. The ketoprofen topical composition of various embodiments has been formulated with drug loading up to 20% using various topically acceptable ingredients and permeation enhancer(s) to improve local bioavailability of drug through skin. The formulation is chemically and physically stable. In some embodiments, (s)-ketoprofen is used as the active ingredient as it is twice as potent as a racemic mixture of ketoprofen, leading to a formulation having one-half the amount of drug loading as a comparably effective racemic mixture. In general, the lower drug loading tends to result in significantly less skin irritation potential.

Another embodiment of the present disclosure is directed to a method of applying an improved ketoprofen formulation for the treatment of inflammation and/or pain associated with arthritis. The method comprises applying a cream comprising any one of the formulations described above to the skin on or around an area of pain or inflammation. In some embodiments, the cream is applied regularly for chronic treatment of arthritis symptoms. In other embodiments, the cream is applied as needed for acute treatment of arthritis pain and inflammation.

### Ketoprofen Formulations for Examples

Four ketoprofen formulations, as shown in Table 1 and Table 2, were tested in the various examples as described below. The ketoprofen formulations were prepared according to the following: liquid paraffin, white petrolatum, cetyl alcohol and stearyl alcohol mixture, polyoxyethylene monostearate and glyceryl monostearate were placed in a first beaker in a water bath at 70° C. and mixed well with a glass rod until all the ingredients were melted to form a clear oleaginous phase. Isopropyl alcohol, isopropyl myristate, oleic acid, methyl paraben, propyl paraben and ketoprofen were placed in a second beaker and mixed well until all the ingredients and drug were dissolved (i.e., drug solution phase). In a third beaker, purified water and triethanolamine or epolamine were combined and mixed well until a clear solution was obtained and then placed in a water bath at 70° C. (aqueous phase). The drug solution (i.e., second beaker) was slowly added to the oleaginous phase (i.e., first beaker) with continuous mixing with a glass rod. The aqueous solution (i.e., third beaker) was then added to the above oleaginous phase and mixed well with a glass rod until a crude emulsion was formed. The crude emulsion was then mixed with a high shear planetary mixer until it reached a congealing temperature of 45-50° C. to form a smooth cream and then set aside to cool down to room temperature. The cream was then transferred to an amber glass jar and tightly sealed.

TABLE 1
Ketoprofen Formulations C and D

| | Formulation-C | Formulation-D |
|---|---|---|
| | (% W/W) | (% W/W) |
| Ingredients | (pH 5.2) | (pH 5.1) |
| Ketoprofen | 9 | 12 |
| Liquid paraffin | 5 | 5 |
| White petrolatum | 5 | 5 |
| Cetyl alcohol-stearyl alcohol mixture | 10 | 10 |
| Polyoxyethylene monostearate | 4 | 4 |
| Glyceryl monostearate | 4 | 4 |
| Isopropyl alcohol | 8 | 8 |
| Isopropyl myristate | 2 | 2 |
| Methyl paraben | 0.1 | 0.1 |
| Propyl paraben | 0.02 | 0.02 |
| Butylated hydroxytoluene | 0.05 | 0.05 |
| Triethanolamine | 1.5 | 2.0 |
| Purified water | 51.3 | 47.8 |
| Total weight, gm | 100 | 100 |

TABLE 2
Ketoprofen Formulations E and F

| | Formulation-E | Formulation-F |
|---|---|---|
| | (% W/W) | (% W/W) |
| Ingredients | (pH 5.1) | (pH 5.2) |
| Ketoprofen | 10 | 10 |
| Liquid paraffin | 5 | 5 |
| White petrolatum | 5 | 5 |
| Cetyl alcohol-stearyl alcohol mixture | 10 | 10 |
| Polyoxyethylene monostearate | 4 | 4 |
| Glyceryl monostearate | 4 | 4 |
| Isopropyl alcohol | 10 | 8 |
| Isopropyl myristate | 2 | 2 |
| Oleic acid | 2 | 0 |
| Methyl paraben | 0.1 | 0.1 |
| Propyl paraben | 0.02 | 0.02 |
| Butylated hydroxytoluene | 0.05 | 0.05 |
| Triethanolamine | 2.5 | 0 |
| Epolamine | 0 | 2.5 |
| Purified water | 45.3 | 47.3 |
| Total weight, gm | 100 | 100 |

### Example 1

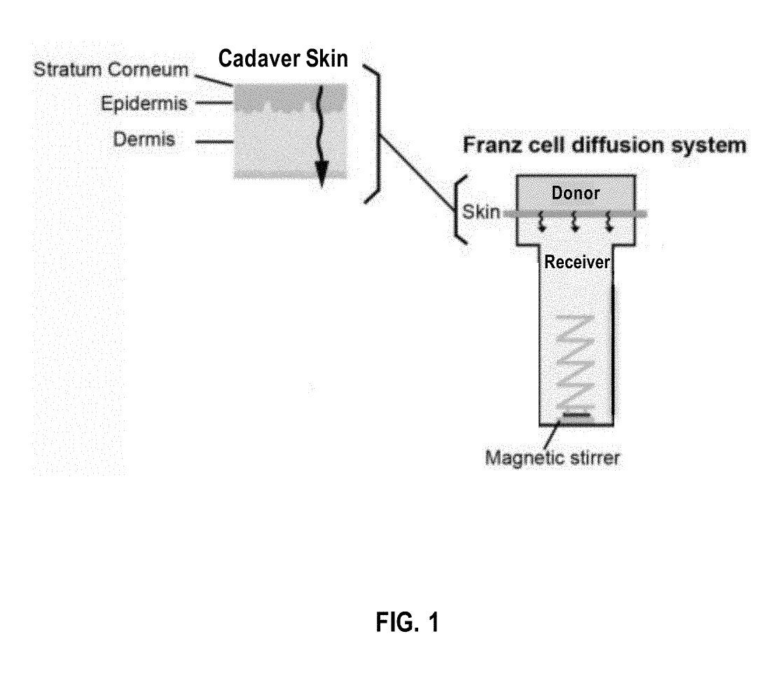

In vitro skin permeation studies of ketoprofen were performed using a Franz cell diffusion system, as shown in FIG. 1. A Franz cell diffusion system includes two chambers: a donor chamber and a receiver chamber with a diffusion area of 0.79 cm². About 3 cm² of cadaver skin (Science Care, Phoenix, Ariz.) was die cut out and an appropriate amount of ketoprofen cream was applied to a 0.79 cm² area on the stratum corneum side of the skin using a metallic template to ensure an exact amount of cream was applied in triplicate diffusion cells. The skin was then positioned on the receiver chamber with the applied cream side facing the donor chamber and an O-ring was placed on the top of the skin. The donor chamber was then positioned on the receiver chamber and tightly clamped. The receiver chamber of the Franz cell diffusion system was filled with phosphate buffered saline (PBS) containing sodium azide (pH 7.4) and a small magnetic stirring bar was placed in the receiver chamber. The assembled Franz cell diffusion system, as shown in FIG. 1, was then positioned on a hot magnetic stirring plate with mixing speed of 200 rpm and the receiver fluid temperature was maintained at 32° C. At a predetermined time, all of the receiver fluid was emptied from the receiver chamber and the receiver chamber was refilled with fresh PBS. The samples were taken at the following intervals: zero hours (to establish the absence of ketoprofen), and then two hours, four hours, and eight hours. The skin samples were assayed for ketoprofen using high performance liquid chromatography (HPLC) with ultraviolet (UV) light detection. At least three diffusion cells were used for each cream formulation tested. The cumulative amount of ketoprofen that permeated as a function of time was determined.

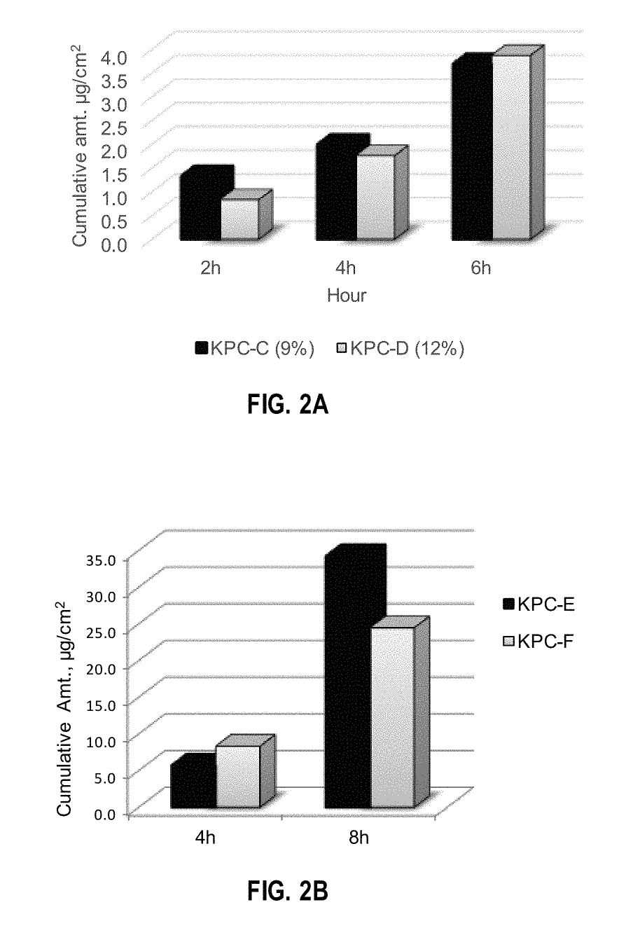

As shown in FIG. 2A, ketoprofen formulations C and D were compared in an in vitro skin permeation study. As shown in Table 1, ketoprofen formulation C (KPC-C) has 9% ketoprofen and ketoprofen formulation D (KPC-D) has 12% ketoprofen. The amount of ketoprofen that permeated the skin at two hours, four hours, and eight hours did not significantly differ between KPC-C and KPC-D, as shown in FIG. 2A. In fact, KPC-D permeated slightly less effectively than KPC-C at two hours and four hours suggesting that the higher level of ketoprofen in KPC-D may slow the permeation process at least at early time points. There was no difference in permeation between KPC-C and KPC-D or even slightly enhanced permeation of KPC-D compared to KPC-C at eight hours suggesting that, at least at later time points, ketoprofen formulations with higher levels of ketoprofen permeate as effectively or more effectively than ketoprofen formulations comprising lower levels of ketoprofen at later time points.

As shown in FIG. 2B, ketoprofen formulations E and F were compared in an in vitro skin permeation study. As shown in Table 2, ketoprofen formulation E (KPC-E) and ketoprofen formulation F (KPC-F) have the same level of ketoprofen (10%) but differ in the buffering agent used. KPC-E comprises 2.5% triethanolamine (with 2% oleic acid as a permeation enhancer) while KPC-F comprises 2.5% epolamine. As shown in FIG. 2B, KPC-F has slightly improved permeation compared to KPC-E at four hours while KPC-E had drastically improved permeation compared to KPC-F at eight hours. These data suggest that a combination of isopropyl myristate/oleic acid and triethanolamine buffer may improve overall skin permeation as compared to isopropyl myristate and epolamine buffer.

### Example 2

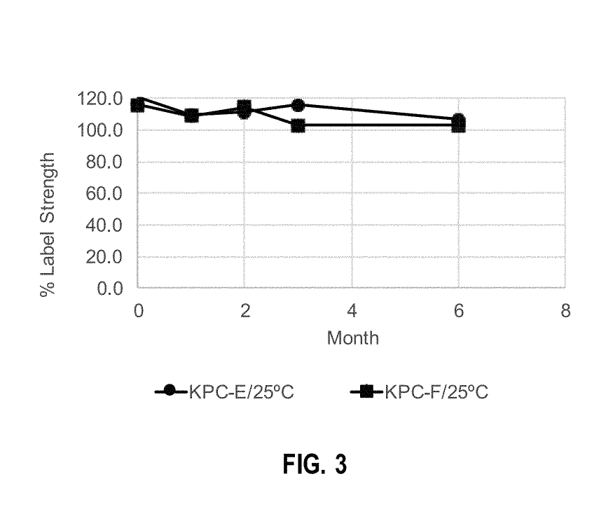

As shown in FIG. 3, the stability of ketoprofen creams, KPC-E and KPC-F, stored at 25° C. in a temperature-controlled oven for six months was tested. KPC-E and KPC-F differ in the buffering agent used as shown in Table 2. Each ketoprofen cream was placed in a scintillation vial wrapped with an aluminum foil and placed at 25° C. in a temperature-controlled oven for six months. The samples were withdrawn at zero months (initial), one month, two months, three months, and six months. Ketoprofen cream was removed from each vial and transferred into a clean new vial and the weight of cream was recorded. About eighteen mL of undiluted methanol was added to the vial containing ketoprofen cream, closed with a cap, and vortexed for about one minute followed by slow mixing in an orbital shaker for about two hours. An aliquot of one mL was filtered and assayed directly for ketoprofen content and impurities using HPLC-UV. The ketoprofen content was reported as a percent label strength. As shown in FIG. 3, there was no significant difference in the stability of ketoprofen formulations, comprising triethanolamine (KPC-E) or epolamine (KPC-F) as a buffering agent, stored at 25° C. for one month, two months, three months, or six months. As shown in FIG. 3, KPC-E and KPC-F both were at about 100% of label strength after storage at 25° C. for six months.

Further, as shown in Table 3, the area percent (area %) for ketoprofen formulations E and F (KPC-E and KPC-F) and related substance (RS) stored at 25° C. over time was determined by HPLC. The area percent and RS measure impurity and/or degradation of the ketoprofen formulations over time after storage at 25° C. As shown in Table 3, a slight increase in RS over six months was observed for both formulations, KPC-E and KPC-F. It appears that KPC-E is slightly better than KPC-F, as KPC-E produced less RS than KPC-F over six months at 25° C.

TABLE 3
Area % for ketoprofen (KP) and related substance (RS) at
25° C. based on HPLC assay

| | KPC-E | KPC-E | KPC-F | KPC-F |
|---|---|---|---|---|
| Month | KP Area % | RS Area % | KP Area % | RS Area % |
| 0 | 99.9 | 0.1 | 99.6 | 0.4 |
| 1 | 99.8 | 0.2 | 99.7 | 0.3 |
| 2 | 99.8 | 0.2 | 99.8 | 0.2 |
| 3 | 99.5 | 0.5 | 99.4 | 0.6 |
| 6 | 99.6 | 0.4 | 98.3 | 1.3 |

### Example 3

As shown in Table 4, skin irritation studies were performed using a 10% KPC-E (Table 2) ketoprofen cream (test or active group), 0% ketoprofen cream (placebo or negative control group), and a 5% sodium dodecyl sulfate (SDS) (positive control group). New Zealand rabbits were used for skin irritation studies. Five rabbits were used and each rabbit received a placebo cream, an active cream, and a positive control cream (5% SDS). Briefly, the rabbit hairs were carefully removed using a trimmer prior to application of the creams. The skin surface was cleaned using rubbing alcohol and dried. The three creams were applied separately to each of the five rabbits and wrapped immediately with breathable gauze tape. After eight hours, the gauze tape was removed and application sites were scored using standard visual score analogs (VAS) for erythema and edema. The following scale was used for skin irritation (both erythema and edema) scoring: 0: none; 1: slight; 2: mild; 3: moderate; and 4: severe. An average score was determined with n=5 as shown in Table 4. There was no significant irritation observed for creams comprising 10% ketoprofen, as shown in Table 4. Further, there was no significant difference in irritation scores between placebo and active creams suggesting ketoprofen is a non-irritant. 5% SDS was used as a positive control which showed slight skin irritation, indicating that experimental parameters were set-up appropriately.

TABLE 4
Skin Irritation Scores for Active and Placebo Ketoprofen Creams

| | Overall | Overall |
|---|---|---|
| | Erythema | Edema |
| Test/Control Group | Score | Score |
| Ketoprofen 10% (Cream) | 0.00 | 0.00 |
| Placebo 0% (Cream) | 0.00 | 0.00 |
| SDS 5% | 0.20 | 0.60 |

The examples described herein show, by way of illustration and not of limitation, specific embodiments in which the subject matter may be practiced. Other embodiments may be utilized and derived therefrom, such that modifications may be made without departing from the scope of this disclosure. This disclosure is intended to cover any and all adaptations or variations of various embodiments, and it will be readily apparent to those of ordinary skill in the art, in light of the teachings of these embodiments, that numerous changes and modifications may be made without departing from the spirit or scope of the appended claims.

What is claimed is:

1. A topical composition which comprises, on a weight basis:
5 to 15% ketoprofen;
5 to 15% of an oily substance;
5 to 15% of a higher alcohol;
0.02 to 0.1% of an antioxidant;
5 to 15% of a surfactant or emulsifying agent;
5 to 15% of solubilizing vehicle;
1 to 10% of a permeation enhancer;
0.01 to 0.5% of a preservative;
1 to 5% of a buffering agent; and
40 to 60% of purified water.

2. The topical composition of claim 1, wherein the composition is a cream.

3. The topical composition of claim 1, wherein the ketoprofen is present substantially in (s)-enantiomeric form.

4. The topical composition of claim 1, wherein the ketoprofen is present in a racemic mixture.

5. The topical composition of claim 1, wherein the antioxidant comprises one or more of: tocopherol and butylated hydroxytoluene.

6. The topical composition of claim 1, wherein the drug solubilizing vehicle comprises one more of: isopropyl alcohol, low molecular weight polyethylene glycol, and isobutyl alcohol.

7. The topical composition of claim 1, wherein the permeation enhancer comprises one or more of: propylene glycol monolaurate, isopropyl myristate, and oleic acid.

8. The topical composition of claim 1, wherein the higher alcohol acts as a stiffening agent.

9. The topical composition of claim 1, wherein the higher alcohol comprises one or more of: cetyl alcohol and stearyl alcohol.

10. The topical composition of claim 1, wherein the preservative comprises one or both of methyl paraben and propyl paraben.

11. The topical composition of claim 1, wherein the buffering agent comprises one or more of: epolamine, triethanolamine, tromethamine, and diethanolamine.

12. The topical composition of claim 1, wherein the surfactant comprises one or more of: polyoxyethylene monostearate, glyceryl monostearate, and glyceryl monooleate.

13. The topical composition of claim 1, wherein the higher alcohol exists in an oleaginous phase comprising one or more hydrocarbons selected from the group consisting of: liquid paraffin, white petrolatum, bees wax, peanut oil, sesame oil, and soybean oil.

14. The topical composition of claim 1, wherein purified water is in an aqueous phase.

15. The topical composition of claim 1, wherein the pH ranges from 3.5 to 7.5.

16. The topical composition of claim 1, wherein the pH ranges from 4.5 to 5.5.

17. The topical composition of claim 1, wherein the oily substance comprises one or more of: liquid paraffin, white petrolatum, peanut oil, sesame oil, soybean oil, bees wax, and synthetic oil.

18. The topical composition of claim 1, wherein the topical composition is used to treat an individual with an inflammatory condition.

19. The topical composition of claim 1, wherein the topical composition is used to treat an individual with arthritis.

20. The topical composition of claim 1, wherein the topical composition is used to treat an individual with pain.
